(19) United States
(12) Patent Application Publication
(10) Pub. No.: US 2026/0232233 A1
(43) Pub. Date: Aug. 13, 2026

(54) MICROFLUIDIC DEVICE AND METHOD FOR MONITORING OR MEASURING ONE OR MORE PARAMETERS OF A FLUID

(71) Applicant: Agency for Science, Technology and Research, Singapore (SG)

(72) Inventors: Yu Chen, Singapore (SG); Ven Wee James Yap, Singapore (SG); Ruiqi Lim, Singapore (SG); Ming-Yuan Cheng, Singapore (SG)

(21) Appl. No.: 19/156,673

(22) PCT Filed: Feb. 20, 2024
(86) PCT No.: PCT/SG2024/050086

§ 371 Date: Aug. 14, 2025

(30) Foreign Application Priority Data
Feb. 28, 2023 (SG) 10202300536X

(51) Int. Cl. A61B 5/1473; A61B 5/145

(52) U.S. Cl. CPC A61B 5/1473; A61B 5/14532

(57) ABSTRACT

The invention is to provide a microfluidic device including a fluid control module configured to be detachably coupled to an external tube, and a replaceable cartridge detachably coupled to the fluid control module. The replaceable cartridge may include a first port arranged in fluidic communication with the fluid control module, a microchannel in fluidic communication with the first port, wherein the fluid control module is configured to manipulate a fluid received from the external tube and/or one or more reagent solutions from the microchannel and subsequently dispose the same as a waste fluid, a second port arranged in fluidic communication with the fluid control module for the waste fluid to flow into the microchannel, and a pressure release valve in fluidic communication with the microchannel, wherein the pressure release valve is configured to regulate an air gap within the microchannel to keep each of the preloaded one or more reagent solutions in the microchannel spaced apart from the waste fluid received from the fluid control module. The invention also provides a method for monitoring or measuring one or more parameters of the fluid.

### CROSS-REFERENCE TO RELATED APPLICATION

This application claims the benefit of priority of Singapore patent application No. 10202300536X, filed 28 Feb. 2023, the content of it being hereby incorporated by reference in its entirety for all purposes.

### TECHNICAL FIELD

Various embodiments relate to a microfluidic device and a method for monitoring or measuring one or more parameters of a fluid received through an external tube, in particular, one or more biomarkers based on blood of a subject received through a cannula.

### BACKGROUND

Significant fluctuations in biomarkers, such as blood glucose and blood gas, may occur among hospitalized patients. Good regulation of such biomarkers is crucial to the patient's morbidity and mortality. To achieve optimal regulation of fluctuating biomarkers, frequent monitoring is required.

For example, in health care settings, the existing clinically validated method of blood glucose monitoring involves a finger prick and a handheld analyser. For example, existing frequent blood glucose monitoring may involve about 48 finger pricks per every 2 days, while even closer blood glucose monitor may require about 96 finger pricks per every 2 days. This is to provide frequent blood glucose monitoring to achieve optimal blood glucose regulation. This tedious and frequent pricking of patient's peripheries results in pain, bruising and skin alterations that add on to the patient's sufferings. Due to the tedious process and other tasks involved in caring for hospitalized patients, glucose monitoring may be delayed, affecting timely treatment, blood glucose regulation and patient outcomes.

Meanwhile, other existing options like continuous glucose monitoring (CGM) measuring interstitial fluid glucose and optical monitoring may not be reliable for acutely ill patients and may require calibration of the devices. CGM may also be inaccurate and the optical monitoring may produce results that vary with skin tone. Most of these existing methods do not provide immediate results.

More than 60% of hospitalized patients require an intravenous (IV) cannula during their hospital stay. The IV cannula may be used for hydration, medication, and various blood testing. To get accurate results for the blood testing through IV cannula, the IV cannula needs to be flushed by saline first to avoid any interference. After that, a section of blood (front-stream blood: 2 to 3 mL) is drawn from IV cannula and discarded due to the potential dilution by the saline. Then a different syringe is used for a blood draw (middle-stream blood) and analysis.

Such an approach consumes a lot of blood for each test and is labour consuming. The manual changing of the syringe may potentially introduce contamination for both the blood sample and the patients.

Thus, there is a need for a miniaturized device to provide a convenient, fast, pain-free, and accurate method to support frequent biomarker (e.g. blood glucose) monitoring of patients, especially critically ill patients, thereby addressing at least the problems mentioned above.

### SUMMARY

According to an embodiment, a microfluidic device is provided. The microfluidic device may include a fluid control module configured to be detachably coupled to an external tube; and a replaceable cartridge configured to detachably couple to the fluid control module. The replaceable cartridge may include a first port arranged to be in fluidic communication with the fluid control module for one or more reagent solutions to flow between the fluid control module and the replaceable cartridge; a microchannel in fluidic communication with the first port, the one or more reagent solutions being initially preloaded in at least one or more parts of the microchannel, wherein the fluid control module may further be configured to manipulate a fluid received from the external tube and/or the one or more reagent solutions received from the replaceable cartridge and subsequently dispose the manipulated fluid and/or the manipulated one or more reagent solutions as a waste fluid; a second port arranged to be in fluidic communication with the fluid control module for the waste fluid to flow into the microchannel, the second port being different from the first port; and a pressure release valve in fluidic communication with the microchannel. The pressure release valve may be configured to regulate an air gap within the microchannel to keep each of the preloaded one or more reagent solutions in the microchannel spaced apart from the waste fluid received from the fluid control module

According to an embodiment, a method for monitoring or measuring one or more parameters of a fluid received through an external tube is provided. The method may include providing a microfluidic device, according to an embodiment and as described herein, detachably coupled to the external tube; regulating one or more reagent solutions between a fluid control module of the microfluidic device and a replaceable cartridge detachably coupled to the fluid control module; manipulating the one or more reagent solutions received from the replaceable cartridge; obtaining and manipulating the fluid from the external tube; and sampling the fluid to monitor or measure the one or more parameters of the fluid. The one or more reagent solutions may be initially preloaded in at least one or more parts of a microchannel of the replaceable cartridge.

### BRIEF DESCRIPTION OF THE DRAWINGS

In the drawings, like reference characters generally refer to like parts throughout the different views. The drawings are not necessarily to scale, emphasis instead generally being placed upon illustrating the principles of the invention. In the following description, various embodiments of the invention are described with reference to the following drawings, in which:

FIG. 1 shows a schematic cross-sectional view of a microfluidic device, according to various embodiments.

FIG. 2 shows an expanded schematic cross-sectional view of a pressure release valve of the microfluidic device of FIG. 1 under low pressure, according to various embodiments.

FIG. 3 shows an expanded schematic cross-sectional view of the pressure release valve of FIG. 2 under high pressure, according to various embodiments.

FIG. 4 shows a flow chart illustrating a method for monitoring or measuring one or more parameters of a fluid received through an external tube, according to various embodiments.

FIG. 5 shows a schematic representation of a miniaturized IV cannula integrated blood sensing system, according to one example.

FIG. 6 shows a schematic cross-sectional view of another exemplary device.

FIG. 7 shows a schematic cross-sectional view of the device of FIG. 6 when sensor flushing is performed.

FIG. 8 shows a schematic cross-sectional view of the device of FIG. 6 when blood pumping is performed.

FIG. 9 shows a schematic cross-sectional view of the device of FIG. 6 when blood sampling is performed.

FIG. 10 shows a schematic cross-sectional view of the device of FIG. 6 when cannula flushing is performed.

FIG. 11 shows a schematic cross-sectional view of the device of FIG. 6 after multiple repetitions of the working procedures.

FIG. 12 shows an exploded schematic view illustrating a wearable microfluidic device, according to one example.

### DETAILED DESCRIPTION

The following detailed description refers to the accompanying drawings that show, by way of illustration, specific details, and embodiments in which the invention may be practiced. These embodiments are described in sufficient detail to enable those skilled in the art to practice the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Embodiments described in the context of one of the methods or devices are analogously valid for the other methods or devices. Similarly, embodiments described in the context of a method are analogously valid for a device, and vice versa.

Features that are described in the context of an embodiment may correspondingly be applicable to the same or similar features in the other embodiments. Features that are described in the context of an embodiment may correspondingly be applicable to the other embodiments, even if not explicitly described in these other embodiments. Furthermore, additions and/or combinations and/or alternatives as described for a feature in the context of an embodiment may correspondingly be applicable to the same or similar feature in the other embodiments.

In the context of various embodiments, the articles “a”, “an” and “the” as used with regard to a feature or element include a reference to one or more of the features or elements.

In the context of various embodiments, the term “about” as applied to a numeric value encompasses the exact value and a reasonable variance.

As used herein, the term “and/or” includes any and all combinations of one or more of the associated listed items.

As used herein, the phrase of the form of “at least one of A or B” may include A or B or both A and B. Correspondingly, the phrase of the form of “at least one of A or B or C”, or including further listed items, may include any and all combinations of one or more of the associated listed items.

As used herein, the expression “configured to” may mean “constructed to” or “arranged to”.

Various embodiments may provide an automatic microfluidic device for in-line blood analysis, more specifically, for blood monitoring through IV cannula. The microfluidic device does not require repeated finger pricking, which is painful for blood collection. In other words, the microfluidic device may include a miniaturized device for frequent and painless blood monitoring for hospitalized patients, with minimum blood consumption. For example, one application may be for frequent monitoring of the glucose levels for critically ill patients. The device may include a microfluidic module with sensors integrated and a replaceable solution cartridge with reagent preloaded for both blood flushing and waste collection.

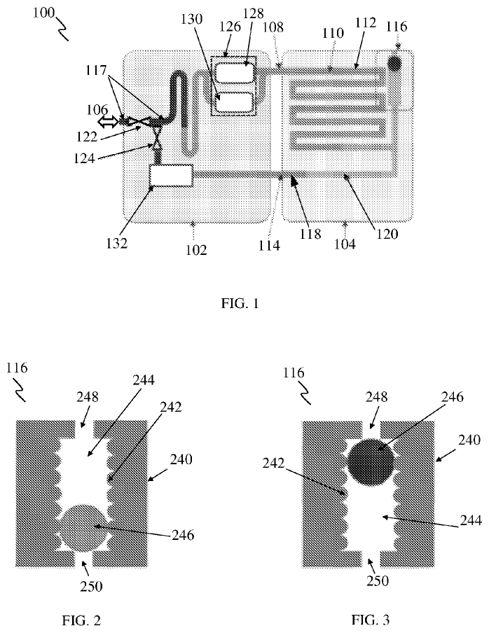

FIG. 1 shows a schematic cross-sectional view of a microfluidic device 100, according to various embodiments. The microfluidic device 100 may include a fluid control module 102 configured to be detachably coupled to an external tube (part of which as indicated by a dual-headed arrow 106); and a replaceable cartridge 104 configured to detachably couple to the fluid control module 102. The replaceable cartridge 104 may include a first port 108 arranged to be in fluidic communication with the fluid control module 102 for one or more reagent solutions 110 to flow between the fluid control module 102 and the replaceable cartridge 104; a microchannel 112 in fluidic communication with the first port 108, the one or more reagent solutions 110 being initially preloaded in at least one or more parts of the microchannel 112; a second port 114 arranged to be in fluidic communication with the fluid control module 102; and a pressure release valve 116 in fluidic communication with the microchannel 112. The fluid control module 102 may be further configured to manipulate a fluid 117 received from the external tube 106 and/or the one or more reagent solutions 110 received from the replaceable cartridge 104 and subsequently dispose the manipulated fluid and/or the manipulated one or more reagent solutions as a waste fluid 118. The second port 114 may be for the waste fluid 118 to flow into the microchannel 112, the second port 114 being different from the first port 108. The pressure release valve 116 may be configured to regulate an air gap 120 within the microchannel 112 to keep each of the preloaded one or more reagent solutions 110 in the microchannel 112 spaced apart from the waste fluid 118 received from the fluid control module 102. By regulating the air gap 120, the pressure release valve 116 may also be configured to regulate each of the preloaded one or more reagent solutions 110 and the waste fluid 118 in the microchannel 112.

In the context of various embodiments, the term “manipulate” associated with the fluid 117 may mean direct or regulate the flow of the fluid, or make measurements to the fluid, or monitor the fluid. The term “manipulate” associated with the one or more reagent solutions 110 may mean direct or regulate the flow of the more reagent solutions or allow the one or more reagent solutions to mix and react with the fluid and/or another one or more reagent solutions.

In various embodiments, the fluid control module 102 may include a first microvalve 122 configured to regulate the fluid 117 between the microfluidic device 100 and the external tube 106; a second microvalve 124 in fluidic communication with the first microvalve 122; and a pump module 126 in fluidic communication with the first microvalve 122 and the second microvalve 124, the pump module 126 being configured to direct the one or more reagent solutions 110 towards or away from the first microvalve 122 and the second microvalve 124. The second microvalve 124 may be arranged between the first microvalve 122 and the pump module 126.

For example, the first microvalve 122 may include a bi-directional microvalve or a multi-way microvalve, and the second microvalve 124 may include a unidirectional microvalve. For example, each of the first microvalve 122 or the second microvalve 124 may be passive or active, normally closed or normally open, mechanical, or non-mechanical. Preferably, each of the first microvalve 122 or the second microvalve 124 may be a normally closed microvalve based on one of the following types: bi-stable, electric, piezoelectric, or thermal.

In various embodiments, the pump module 126 may include a first pump 128 configured to operate in cooperation with the pressure release valve 116 to draw the one or more reagent solutions 110 from the replaceable cartridge 104 and direct the drawn one or more reagent solutions 110 towards the first microvalve 122 and the second microvalve 124; and a second pump 130 arranged fluidically parallel to the first pump 128, the second pump 130 being configured to operate in cooperation with the pressure release valve 116 to draw the one or more reagent solutions 110 away from the first microvalve 122 and the second microvalve 124 and direct the drawn one or more reagent solutions 110 towards the replaceable cartridge 104, thereby allowing the fluid 117 from the external tube 106 to follow the drawn one or more reagent solutions 110 in a direction towards the first port 108 of the replaceable cartridge 104. Effectively, the pump module 126 may provide bi-directional pumping. For example, each of the first pump 128 or the second pump 130 may be passive (e.g. air transfer, capillary, magnetic-driven, chemical-driven, gravity-driven, surface tension) or active, or mechanical (e.g. piezoelectric, electromagnetic, electrostatic, shape memory alloy (SMA), thermo-pneumatic, phase change, ionic conductive polymer film (ICPF), dielectric elastomer film (DEF), diaphragm). Preferably, each of the first pump 128 or the second pump 130 may be an active micropump, or a diaphragm micropump, or a piezoelectric micropump.

The fluid control module 102 may further include a meandering channel arranged between the pump module 126 and the first microvalve 122 and the second microvalve 124. The meandering channel may accommodate a sufficiently long conduit packed within a small space or area in the fluid control module 102 to allow an adequate amount of fluid 117 to be drawn from the external tube 106 into the meandering channel by operating the second pump 130 in cooperation with the pressure release valve 116 to draw the one or more reagent solutions 110 away from the first microvalve 122 and the second microvalve 124, within the meandering channel. With the adequate amount of fluid 117 disposed within the meandering channel, a middle-stream of the fluid 117 may be sampled via the second microvalve 124. In other words, such meandering channel design may advantageously maximize the length of the channel within the fluid control module 102 space, thereby increasing the volume of the fluid drawn to achieve the drawing of the middle-stream fluid 117. In blood monitoring, the meandering channel may facilitate the avoidance of saline-contaminated front-stream blood.

In various embodiments, the fluid control module 102 may further include a check valve arranged to be in fluidic communication with the second port 114 for preventing back-flow of the waste fluid 118.

In various embodiments, the microfluidic device 100 may further include a sensing module 132 configured to receive the fluid 117 via the second microvalve 124 and monitor or measure one or more parameters of the fluid 117. In one example, the sensing module 132 may be integrated in the fluid control module 102. Such integration may provide a form factor and user experience advantage as the microfluidic device 100 may be compact and of a single component/unit for ease of handling by a user. In another example (not shown in drawings), the sensing module 132 may be coupled externally to the fluid control module 102.

The sensing module 132 may include one or more biomarker sensors. For example, the one or more biomarker sensors may include but not be limited to at least one of a blood gas sensor, a blood glucose sensor, a blood pressure sensor, a temperature sensor, or a lactate sensor, an ammonia sensor, or a protein-based detection sensor. The one or more biomarker sensors may be based on one or more of the following working principles: electrochemical, thermal, impedimetric, biomolecular, or colorimetric. Some of the biomarker sensors may be self-powered or passive, while others may require a power source such as a battery to operate. The information acquired by the one or more biomarker sensors may be processed and presented, e.g. using indicators onboard the microfluidic device 100. For example, the fluid control module 102 may include an integrated processor (circuitry board) for controlling the microvalves (e.g. 122, 124), pumps (e.g. 126), readout of the sensing module 132, processing the data and displaying the processed data in a meaningful way (e.g. in a form of an alert system via audio, visual and display panel). Alternatively or additionally, the information may be transmitted to a remote processor for processing and subsequently displayed. This may allow a person of interest (e.g. a nurse or doctor) to be alerted using software applications on a tablet or a mobile device. Electronic and electrical components for the sensing module 132, the microvalves (e.g. 122, 124), pumps (e.g. 126) may be provided on a printed circuit board. The printed circuit board may be arranged or stacked over or under the fluid control module 102.

In various embodiments, the replaceable cartridge 104 may include a third port arranged to be in fluidic communication with the sensing module 132. The fluid control module 102 may further include a micropump configured to operate in cooperation with the pressure release valve 116 to draw another one or more reagent solutions 110 preloaded in the replaceable cartridge 104 and direct the drawn other one or more reagent solutions 110 via the third port to the sensing module 132, the third port being different from the first port 108 and the second port 114. In other words, the micropump may provide a directional pumping for fluid flow from the replaceable cartridge 104 to the fluid control module 102 via the third port.

FIG. 2 shows an expanded schematic cross-sectional view of the pressure release valve 116 under low pressure, while FIG. 3 shows an expanded schematic cross-sectional view of the pressure release valve 116 under high pressure, according to various embodiments. The pressure release valve 116 may include a bi-directional pressure release valve including a body 240 with an interior deformable interface 242 forming a passageway 244 within the body 240; and a movable ball 246 configured to move along the passageway 244 against the interior deformable interface 242 of the body 240 such that the interior deformable interface 242 may be deformable in shape to provide pressure equilibrium 30 within the microfluidic device 100. At one end of the passageway 244, a vent 248 may be provided, while an orifice 250 may be in fluidic communication with the microchannel 112 at the opposite end of the passageway 244. As seen in FIG. 2, when the fluid 117 is not flowing into the microfluidic device 100, e.g. the fluid 117 is flowing out to the external tube 106, low pressure may be experienced in the replaceable cartridge 104 which is sealed. In such a situation, the movable ball 246 moves towards the orifice 250 to block air from entering the microchannel 112 when under low pressure to maintain pressure equilibrium. On the other hand, as seen in FIG. 3, when the fluid 117 is flowing into the microfluidic device 100, more specifically, into the fluid control module 102, the movable ball 246 moves towards the vent 248 under high pressure and the pressure release valve 116 attempts to release the pressure in the replaceable cartridge 104 to maintain pressure equilibrium. The different pressures controlled by the pressure release valve 116, along with the activation of either the first pump 128 or the second pump 130 enables the controlled movements of the fluid 117, the one or more reagent solutions 110 and/or the waste fluid 118.

It should be appreciated that the schematic views of the microfluidic device 100 in FIG. 1 and the pressure release valve 116 in FIGS. 2 and 3 are not limiting and are merely for illustration purposes. For example, variations in layouts, shapes, dimensions, and arrangements are possible without deviating from the intended functions of the microfluidic device 100 and the pressure release valve 116, respectively.

In a case of two or more reagent solutions preloaded in the replaceable cartridge 104, each of these reagent solutions may be arranged as a different segment within the microchannel 112 with an air gap separating a neighbouring reagent solution. The air gaps may be regulated by the pressure release valve 116. In another example, each of these reagent solutions may be in separate chambers disposed in the replaceable cartridge 104, and each of these separate chambers may have at least one port (which may be described in similar context to the first port 108 and/or the second port 114) configured to be removably coupled to the fluid control module 102. In other words, different reagent solutions may enter the fluid control module 102 via different ports, and the number of such ports is not limited to only two ports (e.g. 108, 114 as shown in FIG. 1).

The microfluidic device 100 according to various embodiments may be shaped and dimensioned as a wearable microfluidic device or a portable microfluidic device. For example, the microfluidic device 100 may include a device for blood monitoring through IV cannula. In other words, the device may be for pumping/sampling a testing solution (blood) from IV cannula for automatic on-site analysis. More specifically, the device may include a microfluidic control module (e.g. the fluid control module 102 of FIG. 1), and a replaceable cartridge (e.g. 104) having reagent solution (e.g. 110) and a bi-directional pressure release valve (e.g. 116), wherein the reagent solution and waste (e.g. 118) are separated by an air gap (e.g. 120). The microfluidic control module may be electronically controlled or programmed in a manner to provide an automatic microfluidics module using at least two pumps or micropumps (e.g. 128, 130) for bi-directional pumping and flushing multiple testing and reagent solutions, and at least two valves or microvalves (e.g. 122, 124) for directing the flows of the blood (e.g. the fluid 117) and reagent solutions. The device may further include at least one sensor (e.g. the sensing module 132) for monitoring and measuring purposes. The replaceable cartridge (or interchangeably referred to as a replaceable microfluidic cartridge) may include reagents preloaded in a way that the waste may be collected in the same sealed chamber (i.e. the replaceable cartridge) to occupy the place of the consumed reagent solution such that the total volume of the chamber may be minimized. Waste and multiple reagent solutions may be separated by air gaps in the device to avoid potential contamination. The bi-directional pressure release valve may be integrated to enable repeatable bi-directional blood or reagent solution draw and flushing by repeated pumping in and out of multiple testing and reagent solutions in the sealed chamber. Waste may be collected and transferred to the same chamber of reagent solution to achieve chamber volume savings and weight savings. Advantageously, the sealed chamber helps with the regulation of the pressure and allows fluid movement with the device.

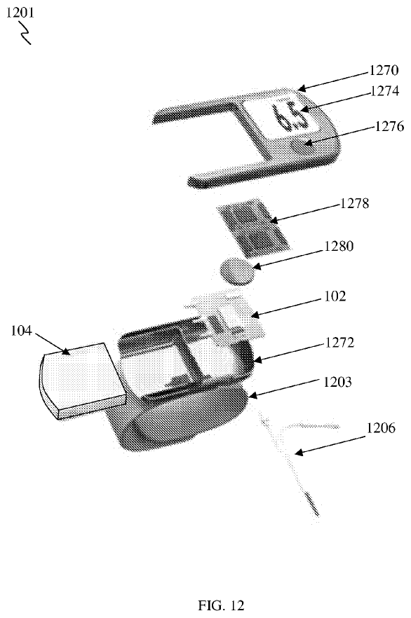

FIG. 12 shows an exploded schematic view 1201 illustrating a wearable microfluidic device, according to one example. As seen in FIG. 12, the wearable microfluidic device may be provided with a wrist strip 1203 coupled to a casing 1270, 1272 which houses a stacked arrangement of the fluid control module 102, a battery 1280, and a printed circuit board 1278 that accommodates the electronic and electrical components for the sensing module 132, the microvalves (e.g. 122, 124), pumps (e.g. 126). A top portion 30 of the casing 1270 may include a display panel 1274 and a visual and/or audio indicator 1276. The casing 1270, 1272 may be designed in a suitable manner to easily receive the replaceable cartridge 104 for coupling with the fluid control module 102, to remove the replaceable cartridge 104 from the casing 1270, 1272, and to provide access between an IV cannula 1206 and the fluid control module 102. The various modules and components may be arranged as depicted in FIG. 12 to minimize the fluidic path length and sample volume requirement for each testing. Such an arrangement or assembly allows for a compact and user-friendly design.

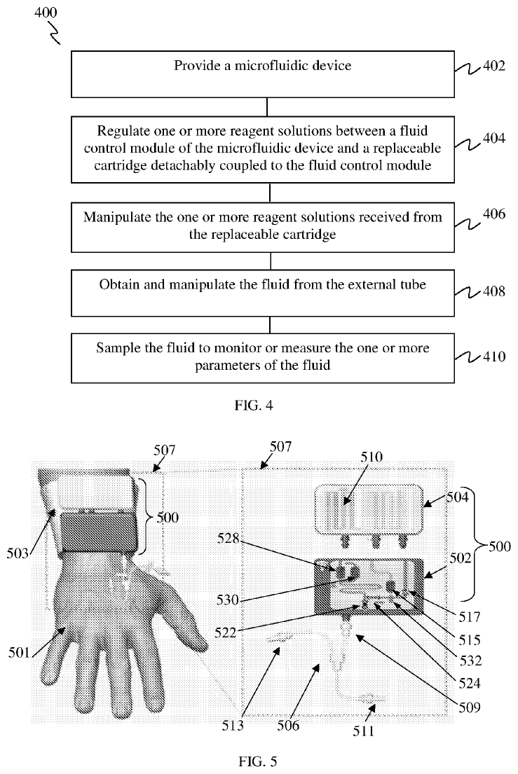

FIG. 4 shows a flow chart illustrating a method 400 for monitoring or measuring one or more parameters of a fluid received through an external tube, according to various embodiments. As seen in FIG. 4, at Step 402, a microfluidic device 100 in accordance with various embodiments detachably coupled to the external tube 106 may be provided. The features of the microfluidic device 100 as described with respect to FIG. 1 may be similarly applicable to the method 400. At Step 404, one or more reagent solutions 110 may be regulated between the fluid control module 102 of the microfluidic device 100 and the replaceable cartridge 104 detachably coupled to the fluid control module 102. The one or more reagent solutions 110 may be initially preloaded in at least one or more parts of the microchannel 112 of the replaceable cartridge 104. At Step 406, the one or more reagent solutions 110 received from the replaceable cartridge 104 may be manipulated. At Step 408, the fluid 117 from the external tube 106 may be obtained and manipulated. More specifically, the flow of the fluid 117 may be manipulated or controlled. At Step 410, the fluid 117 may be sampled to monitor or measure the one or more parameters of the fluid 117.

In various embodiments, regulating the one or more reagent solutions 110 between the fluid control module 102 and the replaceable cartridge 104 at Step 404 may include opening the first microvalve 122 of the fluid control module 102; closing the second microvalve 124 of the fluid control module 102; activating the first pump 128 of the pump module 126 of the fluid control module 102 to direct the one or more reagent solutions 110 to the external tube 106 through the first port 108 of the replaceable cartridge 104 and the fluid control module 102; and activating the pressure release valve 116 of the replaceable cartridge 104 to a low-pressure mode to maintain pressure equilibrium within the replaceable cartridge 104. The low-pressure mode may be as seen in FIG. 2 and pressure equilibrium may be maintained under the low-pressure mode since the one or more reagent solutions 110 is flowing out to the external tube 106.

Manipulating the one or more reagent solutions 110 received from the replaceable cartridge 104 at Step 406 may include closing the first microvalve 122; opening the second microvalve 124 to direct the one or more reagent solutions 110 received from the replaceable cartridge 104 to the sensing module 132 of the fluid control module 102; and subsequently disposing the one or more reagent solutions 110 as the waste fluid 118 from the sensing module 132 to the microchannel 112 through the second port 114 of the replaceable cartridge 104, while maintaining an air gap 120 between the waste fluid 118 and the one or more reagent solutions 110 preloaded in the replaceable cartridge 104. At Step 406, the pressure release valve 116 may be in the low-pressure mode.

Obtaining and manipulating the fluid 117 from the external tube 106 at Step 408 may include opening the first microvalve 122; closing the second microvalve 124; deactivating the first pump 128; activating the second pump 130 of the pump module 126 to direct the fluid 117 from the external tube 106 towards the pump module 126; and activating the pressure release valve 116 to a high-pressure mode to maintain the pressure equilibrium within the replaceable cartridge 104. In other words, the fluid 117 may be directed into part of the meandering channel of the fluid control module 102. The high-pressure mode may be as seen in FIG. 3 and pressure equilibrium may be maintained under the high-pressure mode since the fluid 117 is flowing into the microfluidic device 100.

Sampling the fluid 117 at Step 410 may include closing the first microvalve 122; opening the second microvalve 124; deactivating the second pump 130; and activating the first pump 128 to direct a middle-stream sample of the fluid 117 to the sensing module 132 for monitoring and measurement; and subsequently disposing the middle-stream sample of the fluid 117 as the waste fluid 118 from the sensing module 132 to the microchannel 112 through the second port 114, while maintaining the air gap 120 between the waste fluid 118 and the one or more reagent solutions 110 preloaded in the replaceable cartridge 104. Step 410, the pressure release valve 116 may be in the high-pressure mode.

In various embodiments, in addition to activating the first pump 128 to direct the middle-stream sample of the fluid 117 to the sensing module 132 for monitoring and measurement, the method 400 may further include activating a micropump of the fluid control module 102 to draw another one or more reagent solutions 110 preloaded in the replaceable cartridge 104 and direct the drawn other one or more reagent solutions 110 via a third port of the replaceable cartridge 104 to the sensing module 132 so as to allow the drawn other one or more reagent solutions 110 to react or mix with the middle-stream sample of the fluid 117 for monitoring and measurement.

The method 400 may further include repeating Step 404 to Step 410 to perform repeated monitoring or measuring the one or more parameters of the fluid 117.

In various embodiments, the external tube 106 may include a cannula including one end inserted to a subject and an opposite end detachably coupled to the microfluidic device 100. The fluid 117 may include extracted blood of the subject, and the one or more parameters of the fluid 117 may include or represent one or more biomarkers of the subject. The one or more reagent solutions 110 may include a saline solution, optionally one or more enzymatic solutions, optionally one or more medications, optionally one or more nutrient fluids, and optionally a calibration solution for the sensing module 132.

While the method described above is illustrated and described as a series of steps or events, it will be appreciated that any ordering of such steps or events are not to be interpreted in a limiting sense. For example, some steps may occur in different orders and/or concurrently with other steps or events apart from those illustrated and/or described herein. In addition, not all illustrated steps may be required to implement one or more aspects or embodiments described herein. Also, one or more of the steps depicted herein may be carried out in one or more separate acts and/or phases.

Examples of the microfluidic device 100 (FIG. 1) in the form of an automatic IV cannula-integrated microfluidic blood testing device, which is effectively a middle-stream blood sampling and testing device, and the working procedure of this device corresponding to the method 400 of FIG. 4 will be described below.

The automatic, miniaturized device enables pain-free blood analysis through IV cannula, helps with frequent monitoring of the patient's condition (e.g. less than 1 hour per test), and is user-friendly with minimum (low) blood consumption. The device may be used for critically ill patients who suffer from hypoglycaemia, diabetes ketoacidosis (DKA), hyperosmolar hyperglycemic state (HHS) and/or other critically ill conditions. It may also be used to monitor other biomarkers, such as cortisol levels for mental health.

FIG. 5 shows a schematic representation of the miniaturized IV cannula integrated blood sensing system, according to one example. As seen in FIG. 5, an exemplary device 500 is worn by a subject 501 using a wrist strip 503, with an inset of FIG. 5 denoted by a rectangular dotted box 507 illustrating an interior of the device 500 coupled to an IV cannula 506. One port 509 of the IV cannula 506 is coupled to the device 500, another port 511 may be coupleable to a needle insertable into the subject's vein, and yet another port 513 may be reserved for feeding medications.

The device 500 includes a miniaturized replaceable cartridge 504 preloaded with multiple reagents 510 and a microfluidic control module 502, which includes multiple pumps 528, 530, valves 522, 524, and the sensor 532. In this example, a micropump 515 and a check valve 517 may be provided in the microfluidic control module 502. The replaceable cartridge 504 may also be used as the waste collection chamber by using the air gap to separate the different reagents and the waste. A pressure valve (not shown in FIG. 5) is integrated in the replaceable cartridge 504 to ensure repeatable by-directional blood/solution draw and flushing. The miniaturized replaceable cartridge 504 and the microfluidic control module 502 work incorporation to control the repeated drawing and flushing of the blood cannula with minimized size and weight. The characteristics of the device 500 may be summarized as shown in Table 1 below.

TABLE 1

| Characteristic | Blood Glucose |
|---|---|
| Sampling rate (min/sample) | 5 |
| For acutely ill patients | Yes |
| Testing medium | Blood |
| Comfort level | Painless |
| Accuracy | More than 90% |
| Automatic process | Yes |
| Extra calibration | No |
| Lifetime | 14 days |
| Glucose monitoring frequency | Every 1 to 2 hours |
| (as per doctors' orders) | |

The device 500 may include the same or like elements or components as those of the microfluidic device 100 of FIG. 1, and as such, reference labels with same ending numerals are assigned and the like elements may be as described in the context of the microfluidic device 100 of FIG. 1, and therefore the corresponding descriptions may be omitted here. Essentially, the miniaturized replaceable cartridge 504, the reagents 510, the microfluidic control module 502, the multiple pumps 528, 530, the valves 522, 524, and the sensor 532 may be described in similar context to the replaceable cartridge 104, the one or more reagent solutions 110, the fluid control module 102, the first and second pumps 128, 130, the first and second microvalves 122, 124, and the sensing module 132 of FIG. 1, respectively.

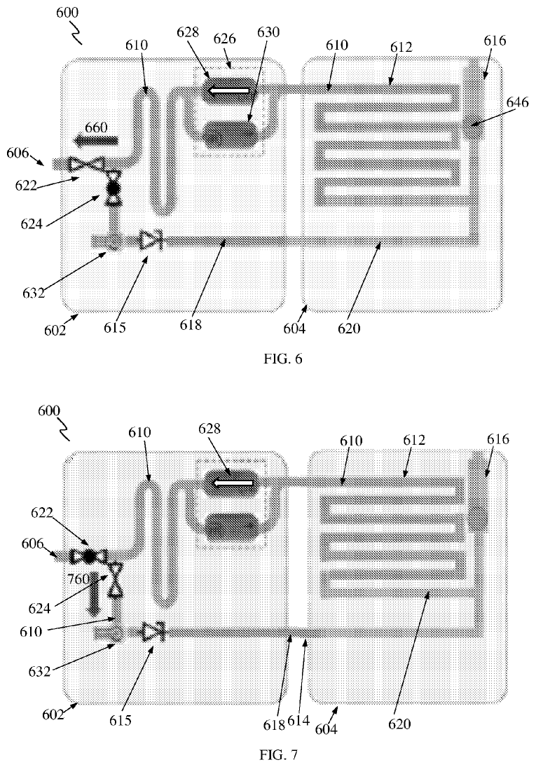

FIG. 6 shows a schematic cross-sectional view of another exemplary device 600. As seen in FIG. 6, a reagent 610 is preloaded into the microchannel 612 of the replaceable cartridge 604, and may be used to flush the cannula 606 (shown in part) or the sensor 632 integrated in the fluid control module 602. The waste 618 is separated by the air gap 620. Other reagents (not shown in FIG. 6), such as a calibration solution for the sensor 632, may be integrated into the replaceable cartridge 604, if needed.

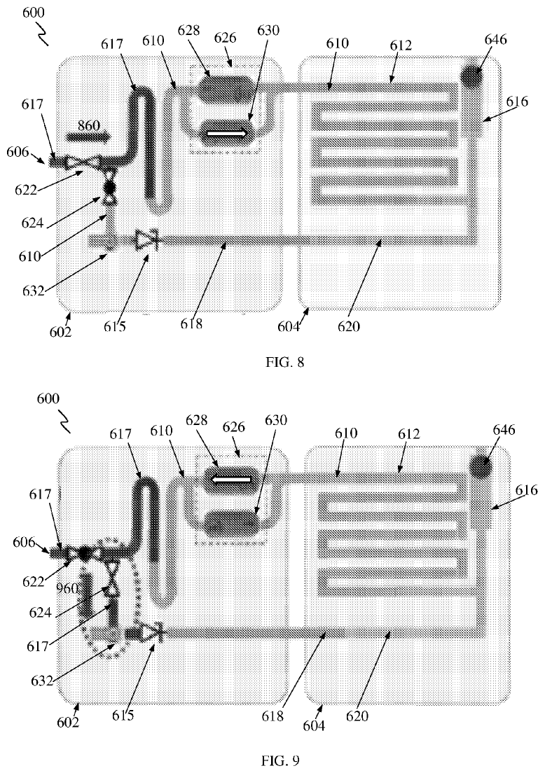

The pressure release valve 616 works with micro pumps 628, 630 of a bi-directional pump module 626 to enable drawing of the blood 617 (see FIGS. 8 to 10) into the replaceable cartridge 604, which may be a sealed chamber, and to flush the reagent 610 from the replaceable cartridge 604 to the cannula 606. The device 600 includes a check valve 615 for preventing backflow of the waste 618. The sensor 632, the micro-pumps 628, 630 and the micro-valves 622, 624 may be electrically controlled and programmed to function.

The device 600 may include the same or like elements or components as those of the microfluidic device 100 of FIG. 1, and as such, reference labels with same ending numerals are assigned and the like elements may be as described in the context of the microfluidic device 100 of FIG. 1, and therefore the corresponding descriptions may be omitted here. Essentially, the reagent 610, the microchannel 612, the replaceable cartridge 604, the cannula 606, the sensor 632, the fluid control module 602, the waste 618, the air gap 620, the pressure release valve 616, the micro pumps 628, 630, the bi-directional pump module 626, the blood 617, the micro-valves 622, 624 may be described in similar context to the one or more reagent solutions 110, the microchannel 112, the replaceable cartridge 104, the external tube 106, the sensing module 132, the fluid control module 102, the waste fluid 118, the air gap 120, the pressure release valve 116, the first and second pumps 128, 130, the pump module 126, the fluid 117, the first and second microvalves 122, 124 of FIG. 1, respectively.

FIGS. 6 to 10 show a working procedure of the device 600, according to one example. FIG. 6 to FIG. 10 may be viewed in sequence.

In FIG. 6, initial flushing may be performed. The device 600 may be connected to the cannula 606. Pump 628 is activated (as denoted by an white arrow) and valve 622 is open. As denoted by a directional arrow 660, the reagent being flushing saline 610 is pumped to the cannula 606 with the help of the pressure release valve 616 where a movable ball 646 is moved to a position within the pressure release valve 616 to maintain pressure equilibrium in view of low pressure forming within the the replaceable cartridge 604.

FIG. 7 shows a schematic cross-sectional view of the device 600 when sensor flushing is performed. As seen in FIG. 7, valve 622 is closed, valve 624 is open, and the flushing saline 610 is pumped to the sensor 632, as denoted by a directional arrow 760. This time, the waste 618 is pushed back to the replaceable cartridge 604 through a waste port 614. The sealed cartridge design enables the migration of the bubble gap (e.g. the air gap 620). The pressure release valve 616 is not activated or affected, since pressure equilibrium may be maintained with low pressure formed within the the replaceable cartridge 604.

FIG. 8 shows a schematic cross-sectional view of the device 600 when blood pumping is performed. As seen in FIG. 8, valve 622 is open, valve 624 is closed and pump 630 is opened or activated (as denoted by an white arrow) to draw blood 617 (front stream) to the fluidic channel (e.g. the meandering channel), as denoted by a directional arrow 860. The pressure release valve 616 is activated where the movable ball 646 is moved to another position within the pressure release valve 616 to release the high pressure formed within the cartridge 604 (sealed chamber). By releasing the high pressure due to the drawn blood 617 entered into the fluid control module 602, pressure equilibrium may be maintained within the replaceable cartridge 604.

FIG. 9 shows a schematic cross-sectional view of the device 600 when blood sampling is performed. As seen in FIG. 9, valve 622 is closed, valve 624 is open, and active pump 628 (as denoted by an white arrow) may pump small amount of blood 617 (such as 50 μL) to the sensor 632 for testing, as denoted by a directional arrow 960. By doing so, middle stream sample with low volume blood may be performed. The waste 618 is pushed into the replaceable cartridge 604 and air gap 620 keeps migrating. The pressure release valve 616 is not activated, since pressure equilibrium may be maintained with high pressure formed within the the replaceable cartridge 604.

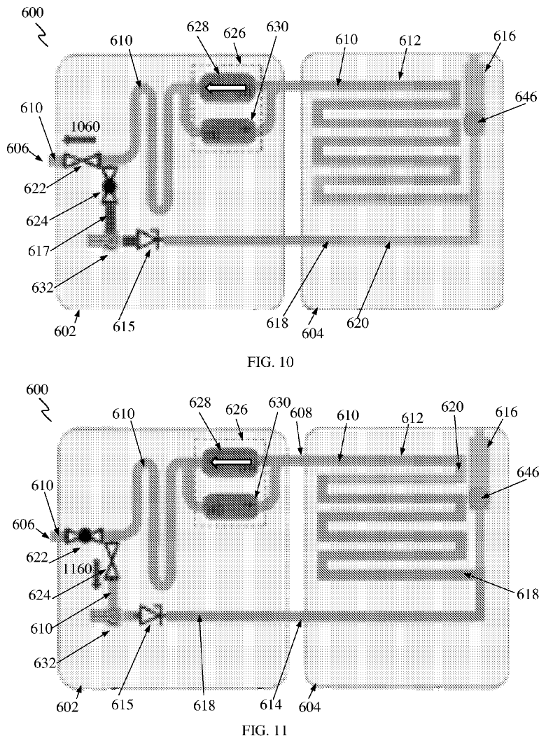

FIG. 10 shows a schematic cross-sectional view of the device 600 when cannula flushing is performed. As seen in FIG. 10, valve 622 is open and valve 624 is closed. With pump 628 activated, the blood 617 is flushed back to the cannula 606 with the help of the pressure release valve 616 where the movable ball 646 is once more moved to the position within the pressure release valve 616 to maintain pressure equilibrium in view of low pressure forming within the the replaceable cartridge 604. In other words, unused blood 617 may be flushed back through the cannula 606 into the subject's body.

The steps as described in FIGS. 6 to 10 may be repeated for blood analysis in a programmed manner. FIG. 11 shows a schematic cross-sectional view of the device 600 after multiple repetitions. With more waste 618 being pumped into the replaceable cartridge 604 and more flushing solution 610 being pumped out, the air gap 620 migrates from the waste port 614 to the reagent port 608. Effectively, there is no contamination and the total size of the replaceable cartridge 604 may be minimized.

The beauty of having the replaceable cartridge with the fluid control module suitable for in-line blood (glucose) testing and waste collection is that the waste may be collected into the same single chamber of the cartridge together with reagent solutions such as a flushing solution or a calibration solution, and enabling low volume blood collection, more specifically, low volume middle stream blood sampling, for recurrent testing. Considering the large volume of waste, it is important to enable this function to minimize the total size of the portable microfluidic device. The pressure release valve plays an important enable pumping of multiple role to repeated bi-directional reagents/solutions/waste (e.g. repeated blood drawing and flushing) in the sealed chamber 30 where air gaps are used to separate multiple reagents and the waste to avoid contamination.

While the invention has been particularly shown and described with reference to specific embodiments, it should be understood by those skilled in the art that various changes in form and detail may be made therein without departing from the spirit and scope of the invention as defined by the appended claims. The scope of the invention is thus indicated by the appended claims and all changes which come within the meaning and range of equivalency of the claims are therefore intended to be embraced.

1. A microfluidic device comprising:
a fluid control module configured to be detachably coupled to an external tube; and
a replaceable cartridge configured to detachably couple to the fluid control module,
wherein the replaceable cartridge comprises:
a first port arranged to be in fluidic communication with the fluid control module for one or more reagent solutions to flow between the fluid control module and the replaceable cartridge;
a microchannel in fluidic communication with the first port, the one or more reagent solutions being initially preloaded in at least one or more parts of the microchannel, wherein the fluid control module is further configured to manipulate a fluid received from the external tube and/or the one or more reagent solutions received from the replaceable cartridge and subsequently dispose the manipulated fluid and/or the manipulated one or more reagent solutions as a waste fluid;
a second port arranged to be in fluidic communication with the fluid control module for the waste fluid to flow into the microchannel, the second port being different from the first port; and
a pressure release valve in fluidic communication with the microchannel, wherein the pressure release valve is configured to regulate an air gap within the microchannel to keep each of the preloaded one or more reagent solutions in the microchannel spaced apart from the waste fluid received from the fluid control module.

2. The microfluidic device as claimed in claim 1, wherein the fluid control module comprises:
a first microvalve configured to regulate the fluid between the microfluidic device and the external tube;
a second microvalve in fluidic communication with the first microvalve; and
a pump module in fluidic communication with the first microvalve and the second microvalve, the pump module being configured to direct the one or more reagent solutions towards or away from the first microvalve and the second microvalve.

3. The microfluidic device as claimed in claim 2, wherein the first microvalve comprises a bi-directional microvalve or a multi-way microvalve, and the second microvalve comprises a unidirectional microvalve.

4. The microfluidic device as claimed in claim 2, wherein the pump module comprises:
a first pump configured to operate in cooperation with the pressure release valve to draw the one or more reagent solutions from the replaceable cartridge and direct the drawn one or more reagent solutions towards the first microvalve and the second microvalve; and
a second pump arranged fluidically parallel to the first pump, the second pump being configured to operate in cooperation with the pressure release valve to draw the one or more reagent solutions away from the first microvalve and the second microvalve and direct the drawn one or more reagent solutions towards the replaceable cartridge.

5. The microfluidic device as claimed in claim 2, wherein the fluid control module further comprises a meandering channel arranged between the pump module and the first microvalve and the second microvalve.

6. The microfluidic device as claimed in claim 1, further comprising a sensing module configured to receive the fluid and monitor or measure one or more parameters of the fluid.

7. The microfluidic device as claimed in claim 6, wherein the sensing module is integrated in the fluid control module.

8. The microfluidic device as claimed in claim 6, wherein the sensing module comprises one or more biomarker sensors.

9. The microfluidic device as claimed in claim 6, wherein the replaceable cartridge comprises a third port arranged to be in fluidic communication with the sensing module; and wherein the fluid control module comprises a micropump configured to operate in cooperation with the pressure release valve to draw another one or more reagent solutions preloaded in the replaceable cartridge and direct the drawn other one or more reagent solutions via the third port to the sensing module, the third port being different from the first port and the second port.

10. The microfluidic device as claimed in claim 1, wherein the pressure release valve comprises a bi-directional pressure release valve comprising:
a body with an interior deformable interface forming a passageway within the body; and
a movable ball configured to move along the passageway against the interior deformable interface of the body such that the interior deformable interface is deformable in shape to provide pressure equilibrium within the microfluidic device.

11. The microfluidic device as claimed in claim 1, wherein the fluid control module further comprises a check valve arranged to be in fluidic communication with the second port for preventing back-flow of the waste fluid.

12. A method for monitoring or measuring one or more parameters of a fluid received through an external tube, the method comprising:
(i) providing a microfluidic device as claimed in claim 1 detachably coupled to the external tube;
(ii) regulating one or more reagent solutions between a fluid control module of the microfluidic device and a replaceable cartridge detachably coupled to the fluid control module, wherein the one or more reagent solutions is initially preloaded in at least one or more parts of a microchannel of the replaceable cartridge;
(iii) manipulating the one or more reagent solutions received from the replaceable cartridge;
(iv) obtaining and manipulating the fluid from the external tube; and
(v) sampling the fluid to monitor or measure the one or more parameters of the fluid.

13. The method as claimed in claim 12, wherein regulating the one or more reagent solutions between the fluid control module and the replaceable cartridge comprises:
opening a first microvalve of the fluid control module;
closing a second microvalve of the fluid control module;
activating a first pump of a pump module of the fluid control module to direct the one or more reagent solutions to the external tube through a first port of the replaceable cartridge and the fluid control module; and
activating a pressure release valve of the replaceable cartridge to a low-pressure mode to maintain pressure equilibrium within the replaceable cartridge.

14. The method as claimed in claim 13, wherein manipulating the one or more reagent solutions received from the replaceable cartridge comprises:
closing the first microvalve;
opening the second microvalve to direct the one or more reagent solutions received from the replaceable cartridge to a sensing module of the fluid control module; and
subsequently disposing the one or more reagent solutions as a waste fluid from the sensing module to the microchannel through a second port of the replaceable cartridge, while maintaining an air gap between the waste fluid and the one or more reagent solutions preloaded in the replaceable cartridge.

15. The method as claimed in claim 14, wherein obtaining and manipulating the fluid from the external tube comprises:
opening the first microvalve;
closing the second microvalve;
deactivating the first pump;
activating a second pump of the pump module to direct the fluid from the external tube towards the pump module; and
activating the pressure release valve to a high-pressure mode to maintain the pressure equilibrium within the replaceable cartridge.

16. The method as claimed in claim 15, wherein sampling the fluid comprises:
closing the first microvalve;
opening the second microvalve;
deactivating the second pump; and
activating the first pump to direct a middle-stream sample of the fluid to the sensing module for monitoring and measurement; and
subsequently disposing the middle-stream sample of the fluid as the waste fluid from the sensing module to the microchannel through the second port, while maintaining the air gap between the waste fluid and the one or more reagent solutions preloaded in the replaceable cartridge.

17. The method as claimed in claim 16, wherein in addition to activating the first pump to direct the middle-stream sample of the fluid to the sensing module for monitoring and measurement, the method further comprises activating a micropump of the fluid control module to draw another one or more reagent solutions preloaded in the replaceable cartridge and direct the drawn other one or more reagent solutions via a third port of the replaceable cartridge to the sensing module so as to allow the drawn other one or more reagent solutions to react with the middle-stream sample of the fluid for monitoring and measurement.

18. The method as claimed in claim 12, further comprising:
repeating (ii) to (v) to perform repeated monitoring or measuring the one or more parameters of the fluid.

19. The method as claimed in claim 12, wherein the external tube comprises a cannula comprising one end inserted to a subject and an opposite end detachably coupled to the microfluidic device, the fluid comprises extracted blood of the subject, and the one or more parameters of the fluid comprises one or more biomarkers of the subject.

20. The method as claimed in claim 12, wherein the one or more reagent solutions comprises a saline solution, optionally one or more enzymatic solutions, optionally one or more medications, optionally one or more nutrient fluids, and optionally a calibration solution.
